(19) United States
(12) Patent Application Publication
(10) Pub. No.: US 2025/0138032 A1
(43) Pub. Date: May 1, 2025

(54) Methods of Predicting Food Allergy

(71) Applicant: National Jewish Health, Denver, CO (US)

(72) Inventors: Donald Leung, Denver, CO (US); Evgeny Berdyshev, Littleton, CO (US); Elena Goleva, Denver, CO (US); Kangmo Ahn, Seoul (KR); Jihyun Kim, Seoul (KR); Byung Eui Kim, Greenwood Village, CO (US)

(21) Appl. No.: 18/933,695

(22) Filed: Oct. 31, 2024

Related U.S. Application Data
(60) Provisional application No. 63/649,156, filed on May 17, 2024.
(60) Provisional application No. 63/594,847, filed on Oct. 31, 2023.

(51) Int. Cl. G01N 33/92; A61B 10/02; G01N 33/68

(52) U.S. Cl. CPC G01N 33/92; A61B 10/02; G01N 33/6869; G01N 2333/54; G01N 2800/24; G01N 2800/50

(57) ABSTRACT

Disclosed herein are methods of treating human infants identified as being at risk of developing a food allergy (FA), as well as methods to prevent the onset of FA symptoms, and/or reduce the severity of FA symptoms, and/or delay the onset of FA symptoms in infants at risk of developing a FA. Further, disclosed herein, are methods of preemptive intervention in human infants at risk of developing a FA. The methods comprise determining the level of one or more unsaturated N-ceramides with C18-sphingosine (N(C18S)CERs), one or more protein-bound OS-ceramides, interleukin-33 (IL-33) or combination thereof in a non-lesional skin sample from the infant.

### CROSS-REFERENCE TO RELATED APPLICATIONS

This application claims the benefit of priority under 35 U.S.C. § 119 (e) to U.S. Provisional Application No. 63/594,847, filed Oct. 31, 2023 and to U.S. Provisional Application No. 63/649,156, filed May 17, 2024. The entire disclosure of each is incorporated herein by reference.

### BACKGROUND

Food allergy (FA) is estimated to affect as many as 8% of children in the United States and 7% in Canada (Fleischer, D. M. et al. J. Allergy Clin Immunol Pract. Vol 9, No 1, November 2020). It has been demonstrated that lipid, metabolomic and protein components of stratum corneum of the skin can help to identify children at risk of future onset of atopic dermatitis (AD) (JACI 2023, 151:1307-1316). Currently, it is not known if stratum corneum components can help identify children and/or infants who are at risk to develop a food allergy (FA). If infants at risk of developing FA before their manifestation of clinical symptoms can be identified, then the application of targeted preemptive interventions and/or therapeutic strategies/measures may prevent and/or reduce FA development and/or severity.

### SUMMARY

One embodiment relates to a method to identify and/or screen a human infant at risk of developing a food allergy (FA), the method comprising: obtaining a skin sample from the infant, wherein the skin sample is a non-lesional skin sample from the infant; determining the level of one or more unsaturated N-ceramides with C18-sphingosine (N(C18S)CERs), one or more protein-bound OS-ceramides, interleukin-33 (IL-33) or combination thereof in the skin sample; and comparing the level of the N(C18S)CER, protein-bound OS-ceramides, IL-33 or combination thereof in the skin sample to a level of the same N(C18S)CER, protein-bound OS-ceramides, IL-33 or combination thereof from a control; wherein an elevated level of the same N(C18S)CER, protein-bound OS-ceramide, IL-33 or combination thereof in the skin sample from the infant as compared to the control identifies the infant as being at risk of developing a FA.

One embodiment relates to a method to prevent the onset of FA symptoms and/or reduce the severity and/or delay the onset of FA symptoms in a human infant at risk of developing a FA, the method comprising: obtaining a skin sample from the infant, wherein the skin sample is a non-lesional skin sample from the infant; determining the level of one or more unsaturated N-ceramides with C18-sphingosine (N(C18S)CERs), one or more protein-bound OS-ceramides, IL-33 or combination thereof in the skin sample; and comparing the level of the N(C18S)CER, protein-bound OS-ceramide, IL-33 or combination thereof in the skin sample to a level of the same N(C18S)CER, protein-bound OS-ceramide, IL-33 or combination thereof from a control; wherein an elevated level of the N(C18S)CER, protein-bound OS-ceramide, IL-33 or combination thereof in the skin sample from the infant as compared to the control identifies the infant as being at risk of developing a FA, wherein the infant identified as being at risk of developing a FA undergoes one or more therapeutic strategies and/or preemptive interventions selected from the group consisting of early introduction of one or more allergenic foods, implementation of a diversified diet comprising one or more allergenic foods, prevention/reduction of household exposure to one or more allergenic foods, administration of a skin barrier enforcing emollient, elimination of S. aureus colonization and infection, allergen-specific immunoglobulin E (IgE) testing to one or more food allergens, skin prick testing (SPT) to one or more food allergens, and an oral food challenge to one or more food allergens.

One embodiment relates to a method of preemptive intervention in a human infant at risk of developing a FA, the method comprising: obtaining a skin sample from the infant, wherein the skin sample is a non-lesional skin sample from the infant; determining the level of one or more unsaturated N-ceramides with C18-sphingosine (N(C18S)CERs), one or more protein-bound OS-ceramide, IL-33 or combination thereof, in the skin sample; and comparing the level of the N(C18S)CER, protein-bound OS-ceramide, IL-33 or combination thereof in the skin sample to a level of the same N(C18S)CER, protein-bound OS-ceramide, IL-33 or combination thereof from a control; wherein an elevated level of the N(C18S)CER, protein-bound OS-ceramide, IL-33 or combination thereof in the skin sample from the infant as compared to the control identifies the infant as being at risk of developing a FA; wherein the infant identified as being at risk of developing a FA undergoes one or more preemptive interventions selected from the group consisting of early introduction of one or more allergenic foods, implementation of a diversified diet comprising one or more allergenic foods, prevention/reduction of household exposure to one or more allergenic foods, administration of a skin barrier enforcing emollient, elimination of S. aureus colonization and infection, allergen-specific immunoglobulin E (IgE) testing to one or more food allergens, skin prick testing (SPT) to one or more food allergens, and an oral food challenge to one or more food allergens.

In one aspect of any of the methods the infant has not been diagnosed as having an atopic disease.

In one aspect of any of the methods, the infant has a family history of an atopic disease.

In one aspect of any of the methods, the one or more N(C18S)CER comprise a long-chain monounsaturated fatty acid. In one aspect, the one or more N(C18S)CER is selected from the group consisting of N24:1(C18S) CER, N26:1(C18S) CER, and combinations thereof.

In one aspect of any of the methods, the protein-bound OS-ceramide is an N32:0O—S-ceramide. In one aspect, the N32:0O—S-ceramide is (N32:0O) (C18S) CER (an OS ceramide with C18-sphingosine and omega-hydroxy 32:0 fatty acid).

In one aspect of any of the methods, the infant identified as being at risk of developing a FA undergoes testing to confirm and/or determine if the infant has a FA.

In one aspect of any of the methods, the infant identified as being at risk of developing a FA further develops atopic dermatitis (AD).

In one aspect of any of the methods, the skin sample is obtained by a skin tape stripping method.

In one aspect, the skin tape stripping method comprises: applying an adhesive tape to a target area of the skin of the infant in a manner sufficient to isolate an epidermal sample adhering to the adhesive tape, wherein the epidermal sample comprises the one or more N(C18S)CERs, one or more protein-bound OS-ceramides, IL-33 or combination thereof from the stratum corneum of the infant, wherein the tape comprises a polymeric adhesive; and extracting the epidermal sample comprising the one or more N(C18S)CERs, one or more protein-bound OS-ceramides, IL-33 or combination thereof adhering to the adhesive tape with a cell scraper comprising thermoplastic elastomer material in a solvent of about 5% to about 30% alcohol in water. In one aspect the skin tape stripping method further comprises determining the expression level of the one or more N(C18S)CERs, one or more protein-bound OS-ceramides, IL-33 or combination thereof in the epidermal sample.

In one aspect of any of the methods, the method further comprises determining thymic stromal lymphopoietin (TSLP) level in the skin sample from the infant and comparing the level to TSLP level in the control sample, wherein the level of TSLP in the infant as compared to the TSLP level of the control is not elevated.

In one aspect of any of the methods, the method further comprises determining the level of TNF-alpha, interleukin-13 (IL-13), interleukin-6 (IL-6) and/or MDC in the infant and comparing the level to levels of TNF-alpha, interleukin-13 (IL-13), interleukin-6 (IL-6) and/or MDC from the control.

### BRIEF DESCRIPTION OF THE DRAWINGS

FIG. 1A is a bar graph demonstrating the pmol/mg protein of NS-ceramides ((N24:1)(C18S) CER and (N26:1)(C18S) CER) in samples from subjects with: NC=control, FA=food allergy, AD=atopic dermatitis, ADFA=atopic dermatitis and food allergy.

FIG. 1B is a schematic of (N24:1)(C18S) CER.

FIG. 1C is a schematic of (N26:1)(C18S) CER.

FIG. 2A is a bar graph demonstrating the pmol/mg protein of protein-bound ceramides (N32:0O) (C18S) CER) in samples from subjects with: NC=control, FA=food allergy, AD=atopic dermatitis, ADFA=atopic dermatitis with food allergy.

FIG. 2B is a schematic of (N32:0O)(C18S) CER.

FIGS. 3A-3D show the stratum corneum unsaturated NS-ceramides as predictors of the future onset of FA and ADFA. Increase in the relative proportions of unsaturated NS-ceramides (N24:1)(C18S) CER and (N26:1)(C18S) CER in the stratum corneum of infants with future development of FA or ADFA (FIG. 3A, FIG. 3B) or combined future FA and ADFA infants (FIG. 3C, and FIG. 3D).

FIGS. 4A-4D show the stratum corneum of future FA infants has increased levels of protein-bound OS-ceramides with ultra-long-chain omega-hydroxy fatty acids. The separation of the future FA group from the healthy and ADFA group is best pronounced for OS-ceramide with C18-sphingosine (N32:0O)(C18S) CER (FIG. 4B). Note the decrease in OS-CER levels in infants with future AD (FIG. 4A-FIG. 4D).

FIGS. 5A and 5D show unsaturated sphingomyelin species are not increased in the stratum corneum of infants with future FA. The increase in absolute levels (FIG. 5A) and relative proportions (FIG. 5D) of 24:1- and 26:1-sphingomyelin molecular species in the SC of infants with future AD but not FA or ADFA. Note that the development of FA cancels the increase in 26:1-SM in the ADFA group.

FIG. 5B is a schematic of a 24:1-sphingomyelin.

FIG. 5C is a schematic of a 26:1-sphingomyelin.

FIGS. 6A-6C show TSLP and IL-33 levels in the stratum corneum are unique for infants with future FA. (FIGS. 6A-6B) TSLP is not increased in the skin of infants with future FA but increased in the skin of future AD and ADFA infants. IL-33 is increased in the skin of future ADFA infants but not in the skin of future AD infants. No selectivity in other cytokines measured was found. FIG. 6C shows that IL-33 levels separate infants with future FA irrespective of AD status.

FIG. 7 shows a logistic regression analysis that reveals the strong combinatorial power of STS cytokines and lipids to predict the future onset of FA by 24 months of age, irrespective of AD status. The sole and combined effects of cytokines and lipids were evaluated by the estimates of odds ratios (ORs) from the logistic regression models. All study participants were included in the analysis. Dotted line—2× fold change.

### DETAILED DESCRIPTION

It has been demonstrated that lipid, metabolites and protein components of stratum corneum (SC) of the skin can help to identify children at risk of future onset of atopic dermatitis (AD) (JACI 2023, 151:1307-1316). Currently, it is not known if stratum corneum components can help identify children and/or infants who are at risk to develop a food allergy (FA).

Atopic dermatitis (AD) is the most common inflammatory skin disease worldwide affecting nearly 30% of children. Very often, AD progresses into FA, asthma, and allergic rhinitis that can persist over the entire human life. Collectively, this transition of atopic diseases is called the atopic march. Atopic diseases are impossible or very hard to cure and impose a huge burden on society. Therefore, finding the early biomarkers of the future onset of atopic diseases in newborns, such as FA, opens a window for preemptive interventions that can potentially decrease the severity or totally halt the onset of atopic diseases. Non-invasive collection of biological material to detect biomarkers is critical taking into account the age of infants (early weeks or months), and human SC represent the ideal material that is absolutely benign to collect using skin tape stripping (STS) procedure even on newborns.

The inventors have undertaken a study as disclosed herein wherein newborn infants of Asian ethnicity were clinically followed for up to 2 years of age. A non-invasive approach using STS was used to collect SC skin samples from the forearm at the age of 2 months when none of the infants demonstrated any signs of FA, AD and/or other atopic diseases. STS samples were analyzed by liquid chromatography electrospray ionization tandem mass spectrometry (LC-MS/MS) for protein-bound ceramides (also referred to as “OS-CER”) that are critical for the scaffold formation during SC maturation and epidermal barrier formation. The same STS samples were further analyzed by LC-MS/MS for free ceramides that comprise major barrier-forming lipid layer components responsible for skin barrier function and are laid upon the scaffold formed by protein-bound ceramides. Both protein-bound and free ceramides were upregulated. The free ceramides were determined to be unsaturated N-ceramides with C18-sphingosine ((C18S) CERs) and with N-type (i.e., no alpha-hydroxy group) long-chain monounsaturated fatty acids. In a preferred embodiment, these ceramides are N24:1(C18S) CER and N26:1(C18S) CER. The “(C18S) CER” specifies a particular sphingoid base with 18 carbon and 1 double bond.

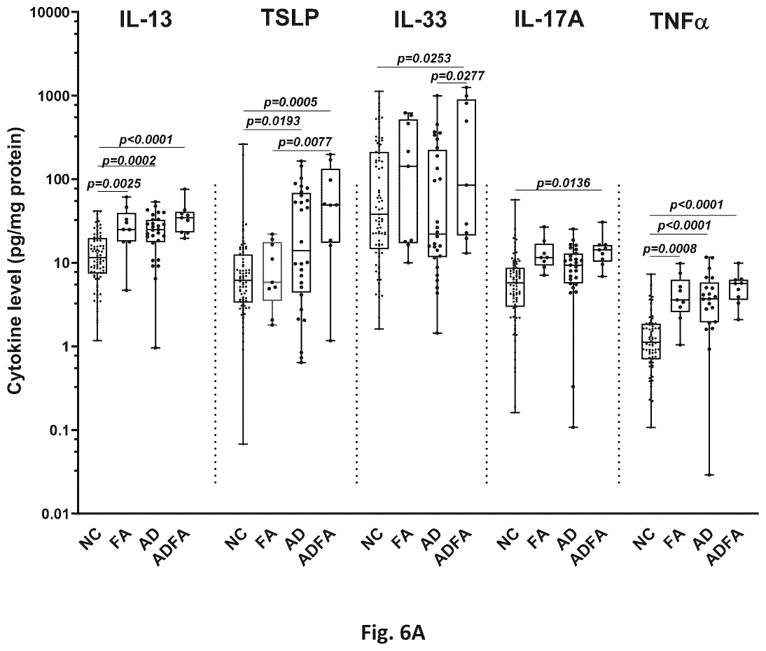

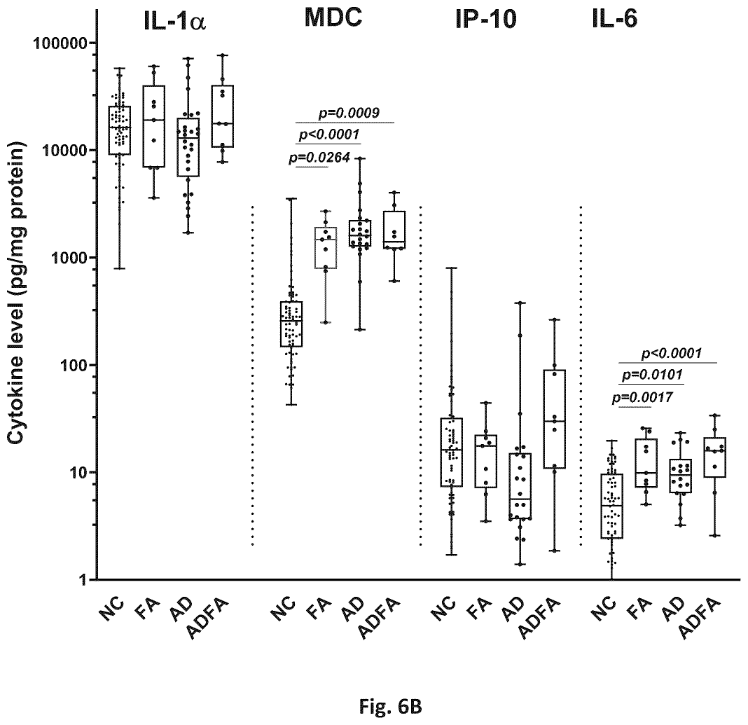

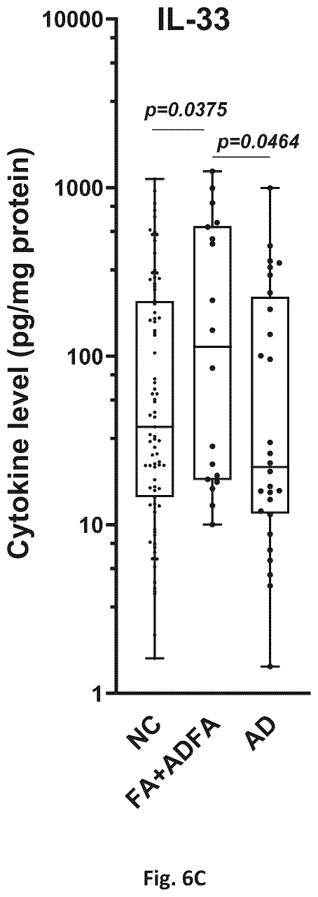

Further, cytokines are well known to regulate lipid metabolism (Kitaura J, Murakami M. Positive and negative roles of lipids in mast cells and allergic responses. Curr Opin Immunol 2021; 72:186-95), and Type 2 cytokines such as interleukin (IL)-4 and -13 are critical for IgE synthesis and lipid changes in the SC in AD (Berdyshev E, et al. Stratum corneum lipid and cytokine biomarkers at age 2 months predict the future onset of atopic dermatitis. J Allergy Clin Immunol 2023; 151:1307-16; Danso M O, van Drongelen V, Mulder A, van Esch J, Scott H, van Smeden J, et al. TNF-alpha and Th2 cytokines induce atopic dermatitis-like features on epidermal differentiation proteins and stratum corneum lipids in human skin equivalents. J Invest Dermatol 2014; 134:1941-50). Keratinocyte-produced alarmin, thymic stromal lymphopoietin (TSLP), has been demonstrated to be a predictive biomarker in the SC of future AD infants (Kim J., et al. Epidermal thymic stromal lymphopoietin predicts the development of atopic dermatitis during infancy. J Allergy Clin Immunol 2016; 137:1282-5 e4), and keratinocyte-generated IL-33 (but not TSLP) has been shown to play a role in IgE-mediated gut mast cell activation and FA initiation in mice (Leyva-Castillo J M., et al. Mechanical Skin Injury Promotes Food Anaphylaxis by Driving Intestinal Mast Cell Expansion. Immunity 2019; 50:1262-75 e4.; Han H, et al. IL-33 promotes gastrointestinal allergy in a TSLP-independent manner. Mucosal Immunol 2018; 11:394-403). Therefore, the inventors examined Type 2 SC cytokines to differentiate skin cytokine dysregulation in FA versus AD. As demonstrated in the examples and figures provided herein, several cytokines (TNF-α, IL-6, IL-13, macrophage-derived chemokine (MDC)) were equally elevated in future FA, AD, and ADFA groups (FIG. 6A and FIG. 6B). IL-1α and interferon gamma-induced protein 10 (IP-10) were not increased compared to healthy infants (FIG. 6B). TSLP was not elevated in future FA infants but was elevated in the SC of future AD infants and in future ADFA infants (FIG. 6A). This correlates with the known role of TSLP in the development of AD (Luo J, et al. The Role of TSLP in Atopic Dermatitis: From Pathogenetic Molecule to Therapeutical Target. Mediators Inflamm 2023; 2023:7697699). IL-33 was not elevated in the skin of future AD infants, but demonstrated moderate elevation in the skin of future FA-only infants and was statistically significantly higher (P<0.05) in SC of the future ADFA group in comparison to either healthy or future AD infants (FIG. 6A). Combining future FA and ADFA infants into one group led to the complete separation of future FA infants, irrespective of their AD status, for SC IL-33 levels as compared to healthy infants (FIG. 6C). Models of allergic inflammation have shown distinct requirements for IL-33 in skin sensitization and intestinal manifestations of FA independent of TSLP (Leyva-Castillo J M., et al. Mechanical Skin Injury Promotes Food Anaphylaxis by Driving Intestinal Mast Cell Expansion. Immunity 2019; 50:1262-75 e4; Wang Y H. Developing food allergy: a potential immunologic pathway linking skin barrier to gut. F1000Res 2016; 5). Notably, in future ADFA, the increase in both TSLP and IL-33 was detected in SC. This is supported by experimental studies in animals that have shown TSLP promoting the expression of IL-33 receptors on many inflammatory cell types, allowing IL-33 to amplify type 2 immune responses (Han H, et al. The atopic march: current insights into skin barrier dysfunction and epithelial cell-derived cytokines. Immunol Rev 2017; 278:116-30). Thus, TSLP and IL-33 upregulation differentiates future FA, AD, and ADFA groups.

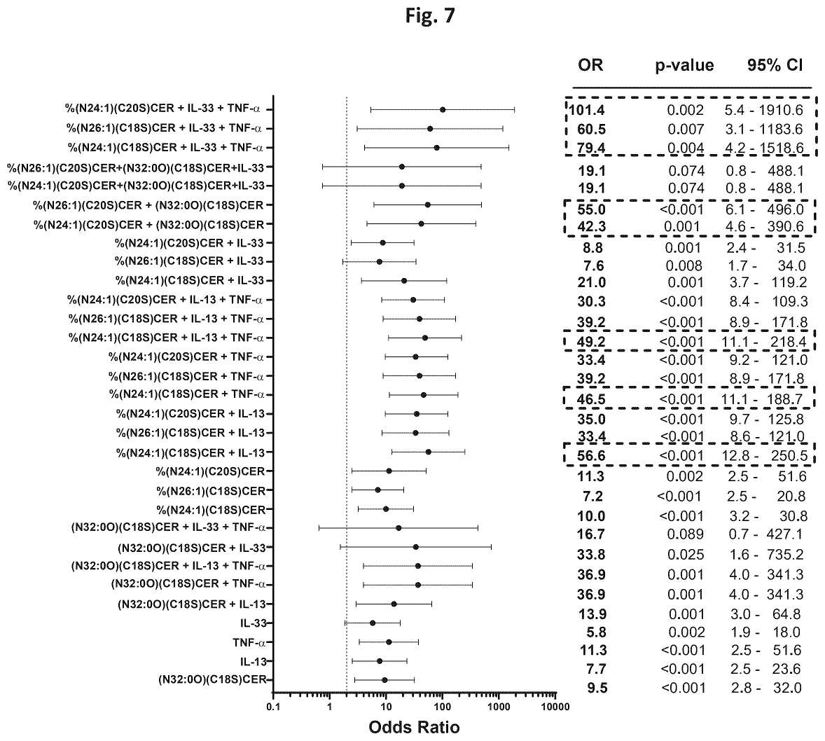

Additionally, the inventors evaluated the power of identified lipid and cytokine parameters to predict the future onset of FA irrespectively of the future AD status using a logistic regression approach. FIG. 7 demonstrates that unsaturated NS-ceramide species as well as protein-bound OS-ceramides (the most distinctive OS-ceramide molecular species chosen), have strong predicting power on their own (odds ratios (OR) s from 7.2 to 11.3). Furthermore, altered lipid profiles in combination with IL-33 (FA-specifically upregulated cytokine) and TNFα (non-specifically upregulated cytokine) substantially increased the possibility of predicting FA, with OR reaching a value above 100 (FIG. 7).

The findings herein provide a roadmap for preemptive intervention in infants at risk of future development of FA as well as future development of FA and AD together.

AD usually precedes other atopic diseases such as FA, asthma, and allergic rhinitis. However, the inventors have now determined that early prediction of future FA and/or FA with AD can also be determined. These results highlight novel skin biomarkers of the future FA as well as FA with AD onset that, when combined, provide an unprecedented power of prediction and open the field of precision medicine in FA as well as AD.

The findings presented herein are the first indication that changes in the SC lipidome precede future onset of FA. Importantly, the inventors have identified lipid and cytokine signatures that are unique for the future onset of FA and AD and can be used as predictive biomarkers. Changes in lipid composition identified in the skin of future AD (decrease in protein-bound ceramides and increase in unsaturated SM species) and FA infants (increase in monounsaturated NS ceramides) suggest the critical contribution of the skin barrier in the initiation of allergic sensitization (Han H, et al. The atopic march: current insights into skin barrier dysfunction and epithelial cell-derived cytokines. Immunol Rev 2017; 278:116-30; Goleva E, et al. Epithelial barrier repair and prevention of allergy. J Clin Invest 2019; 129:1463-74; Akdis C A., et al. Allergy: Mechanistic insights into new methods of prevention and therapy. Sci Transl Med 2023; 15: eadd2563). A detrimental role of fatty acid unsaturation for skin barrier function is supported by recent observations in an experimental SC substitution model demonstrating increased epidermal permeability and transepidermal water loss by ceramides with monounsaturated fatty acids (Mojumdar E H, et al. Monounsaturated fatty acids reduce the barrier of stratum corneum lipid membranes by enhancing the formation of a hexagonal lateral packing. Langmuir 2014; 30:6534-43). Importantly, the inventor's SC cytokine analysis demonstrates selective activation of epidermal alarmins in the skin of future AD (TSLP), ADFA (TSLP, IL-33), and future FA children (IL-33). These epithelial cell-derived cytokines license dendritic cells to activate type 2 responses, but also act on basophils, eosinophils, mast cells and innate lymphoid cells to initiate and maintain allergic inflammation. As there are targeted biologics currently under development for IL-33 and are now available to TSLP and IL-4/IL-13 (Beck L A, et al. Type 2 Inflammation Contributes to Skin Barrier Dysfunction in Atopic Dermatitis. JID Innov 2022; 2:100131; Bieber T, et al. Atopic dermatitis: pathomechanisms and lessons learned from novel systemic therapeutic options. J Eur Acad Dermatol Venereol 2022; 36:1432-49; Wang Z, Tang N. Unpacking the complexity of nuclear IL-33 (nIL-33): a crucial regulator of transcription and signal transduction. J Cell Commun Signal 2023; 17:1131-43) (the latter is currently approved down to 6 months of age), early identification of infants at risk for future AD or FA development opens possibilities for targeted interventions to prevent and circumvent their clinical disease.

An “individual” is a vertebrate, such as a mammal, including without limitation a human. Mammals include, but are not limited to, farm animals, sport animals, companion animals (such as dogs and cats), primates, mice and rats. The term “individual” can be used interchangeably with the term “animal”, “subject” or “patient”.

In one aspect of the invention, the subject is human. In one aspect, the subject is a child (between two years of age and eighteen years of age). In another aspect, the subject is an infant. Infant as used herein is defined as up to two years (24 months) of age. In a preferred embodiment, the infant is less than one year old.

An asymptomatic subject is a subject that is not producing or showing symptoms of an allergic disease. For example, an AD asymptomatic subject is a subject that is not producing or showing symptoms of AD such as, itching, red patches on the skin (especially on the hands, feet, ankles, wrists, neck, upper chest, eyelids, inside the bend of the elbows and knees, face and scalp); small, raised bumps which can leak fluid and crust over when scratched; thickened, cracked, dry, scaly skin; and raw, sensitive, swollen skin from scratching. Most often, AD begins before age 5 and may persist into adolescence and adulthood. For some AD subjects, it flares up periodically and then clears up for a time. An FA asymptomatic subject, is a subject that is not producing or showing any symptoms of a FA such as, severe nausea or vomiting, diarrhea, stomach cramps or stomach pain, red-itchy rash (hives), swelling of the face, eczema, itching or swelling of the lips, tongue or mouth, itching or tightness in the throat, difficulty breathing, wheezing, colic, blood in subject's stool, poor growth, drop in blood pressure, and/or a runny or stuffy nose.

The term “sample” or “patient sample” or “subject sample” or “test sample” can be used generally to refer to a sample of any type which contains products that are to be evaluated by the present methods, including but not limited to, a skin sample including a skin epidermal sample, a skin sample from the stratum corneum, a tissue sample and/or a bodily fluid sample. The stratum corneum (SC) is the outer layer of the skin (epidermis). It serves as the primary barrier between the body and the environment. The SC is multi layered and is composed of dead, anucleated, flattened corneocytes. The stratum corneum has a thickness between 10 and 40 μm and can contain about 15-20 layers. In one aspect of the invention, the skin sample comprises skin layers 1, 2, and/or the sum of layers 1 and 2 from the SC. In yet another aspect of the invention, the skin sample comprises skin layers 3, 4, and/or the sum of layers 3 and 4 from the SC. In still another aspect, the skin sample comprises layers 5, 6 or 15, 16 and/or the sum of layers 5 and 6 or 15 and 16 from the SC. In one aspect, the skin sample is taken from non-lesional skin (i.e., skin that appears healthy or normal looking, without any rash).

In one aspect, the control used herein is/are an established level(s) of the ceramides disclosed herein (such as the N(C18S)CERs disclosed herein) determined from one or more subjects that have been determined to be healthy. Healthy subjects are non-atopic subjects that do not have a family history of atopic diseases nor are producing or showing any symptoms of having any atopic diseases including FA and/or AD.

In one aspect, the food allergy includes but is not limited to a peanut allergy, a diary allergy (such as a milk allergy), an egg allergy, a wheat allergy, a tree nut allergy and/or a combinations thereof. Allergenic foods include but are not limited to peanuts, tree nuts, milk, wheat, and eggs.

In one aspect of any of the embodiments related to a method, the subject identified as at risk of developing one or more food allergies undergoes one or more therapeutic strategies/measures and/or targeted preemptive interventions to prevent and/or delay the onset of one or more food allergy symptoms and/or reduce the severity of one or more food allergy symptoms. Such strategies or interventions include early introduction of one or more allergenic foods, implementation of a diversified diet comprising one or more allergenic foods, prevention/reduction of household exposure to one or more allergenic foods, administration of a skin barrier enforcing emollient, elimination of S. aureus colonization and infection, allergen-specific immunoglobulin E (IgE) testing to one or more food allergens, skin prick testing (SPT) to one or more food allergens, and an oral food challenge to one or more food allergens.

In regard to the early introduction of one or more allergenic foods, the subject is administered one or more allergenic foods when the subject is younger than 5 months of age.

In regard to administration of a skin barrier enforcing emollient, a lipid emollient cream/lotion is administered to the skin of the subject. Such cream or lotion can help to reduce eczema and/or the presentation of eczema.

In one aspect of any of the embodiments related to a method, the subject identified as at risk of developing a FA is administered composition comprising an antihistamine, and epinephrine.

In one aspect of any of the embodiments related to a method, the subject identified as at risk of developing a FA and further develops AD is administered a composition comprising a compound selected from the group consisting of corticosteroids, leukotriene antagonists, anti-cytokine antibodies, anti-cytokine receptor antibodies, anti-IgE antibody, anti-interleukin 14 (IL14) antibodies, anti-interleukin 13 (IL13) antibodies, anti-interleukin 33 (IL33) antibodies, JAK kinase inhibitors, JAK/STAT inhibitors, antibiotics, a phosphodiesterase inhibitor, a cream comprising filaggrin or components thereof, ceramide rich emollients, and combinations thereof. In one aspect, the composition is administered to the subject by an administration route selected from the group consisting of local administration, topical administration, and injection.

The cells (comprising the N(C18S)CERs) in the skin sample for example are not necessarily of the same type, although purification methods can be used to enrich for the type of cells that are preferably evaluated. Cells can be obtained, for example, by a tape stripping method (also referred to as “skin taping”), scraping of a tissue, and processing of a tissue sample to release individual cells and/or the N(C18S)CERs. In one aspect, an adhesive tape is applied to a target area of the skin of the subject in a manner sufficient to isolate an epidermal sample adhering to the adhesive tape. In one aspect, the epidermal sample comprises cells (comprising N(C18S)CERs) from the stratum corneum of the subject. In one aspect, the tape comprises a polymeric adhesive. In regards to removal or extraction of cells and thus lipids from the skin sample, the skin material can be removed from tape strips by scraping it out manually by a polymeric scraper, a scraper comprising thermoplastic elastomer material, or using an automated tool with rubber or Teflon scraper of any form, in an alcohol solvent (such as 1-30%). The alcohol can be methanol, ethanol, butanol or isopropanol. Alternatively, the tape strip material can be removed from the tape strips by sonication in an alcohol solution as noted above. Alternatively, skin lipids, and proteins, can be directly extracted from tape strips by supercritical extraction. In one aspect of the invention, the skin sample can be taken from one or more regions of the subject's body, including lesional and/or non-lesional skin. In yet another aspect, one or more skin samples can be obtained and the one or more N(C18S)CERs can be analyzed by liquid chromatography electrospray ionization tandem mass spectrometry (LC-MS/MS) LC/MS/MS.

Methods to measure protein and/or lipid expression levels generally include, but are not limited to: mass spectrometry, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), U-PLEX® Biomarker Group 1 (Human) Multiplex Assays (Meso Scale Discovery (MSD), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al., 1993, Anal. Biochem. 212:457; Schuster et al., 1993, Nature 365:343). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR).

As used herein, an elevated (or increased) level (such as a protein or lipid expression level) means that the level is statistically higher in comparison to the same levels (such as the same proteins) from control subjects. An increase level is greater than 2-fold, 2.1-fold, 2.2-fold, 2.3-fold, 2.4-fold, 2.5-fold, 2.6-fold, 2.7-fold, 2.8-fold, 2.9-fold or about 3-fold increase (or about a 200% to 300% increase) as compared to control levels. A decreased level means that the level is statistically lower in comparison to the same levels from control subjects. A decrease level is about a 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold or about a 3-fold decrease (or about a 150%-300% decrease) as compared to control levels.

The following examples are provided for illustrative purposes and are not intended to limit the scope of the invention as claimed herein. Any variations which occur to the skilled artisan are intended to fall within the scope of the present invention.

### EXAMPLES

### Example 1

This example demonstrates that early life STS lipidomic analyses can identify infants at risk of future development of FA through the elevation of unsaturated N-ceramides with C18-sphingosine (N(C18S)CER) levels.

### Methods

Newborns (n=119) with and without a family history of atopic diseases (risk group, n=79, control group, n=40) were enrolled. Skin tape strips (STS) were collected from the volar area of the forearm at the age of two months before any signs of clinical FA or AD, and infants were clinically monitored until their age of two years. STS were subjected to lipidomic analyses by the LC/MS/MS.

### Skin Tape Stripping, Protein Extraction and Mass Spectrometry Analysis

A total of 4 consecutive D-SQUAME® tape strips (22 mm diameter, CuDerm, Dallas, TX, USA) were collected on the volar surface of right forearm at ages of 2 months. On application of the first tape disc, 4 marks were placed around the disc with pen so that subsequent discs could be applied to the same location. Each tape disc was placed adhesive side up in its own 6-well plate and then frozen at −80° C. Strips 5 and 6 were processed for the extraction of free lipids, total sample protein estimation, protein hydrolysis, and re-extraction of protein-bound ceramides as described herein and in Berdyshev, E., et al., Allergy 2022. ³¹ Liquid chromatography tandem mass spectrometry of lipids was performed using AB Sciex 6500 QTRAP mass spectrometer with Shimadzu Nexera X2 UHPLC front end and Ascentis Express RP-Amide column (2.7 μm 2.1×50 mm) with gradient elution from methanol:water:formic acid (50:50:0.5, 5 mM ammonium formate) to methanol:chloroform:water:formic acid (90:10:0.5:0.5, 5 mM ammonium formate).

All lipid standards were from Avanti Polar Lipids (Birmingham, AL). All ceramide molecules were detected in positive ions mode as a transition from the molecular ion to corresponding sphingoid base minus 2H₂O product ion. Lipid absolute quantitation was achieved using either standard curves of responses of variable amounts of analytes versus fixed amounts of the internal standards or semi quantitatively by comparing lipid signal areas against the signal of corresponding internal standard. Quantitation of NS-ceramides with C18-sphingosine and long-chain monounsaturated fatty acid N26:1 (N26:1(C18S) CER) was achieved using a coefficient obtained from the standards curve for N24:1(C18S) CER. Identification of sphingomyelins was performed in positive ions, and quantitation of sphingomyelins was achieved as a transition from the molecular ions to the m/z 184 (phosphocholine) using N-(dodecanoyl)-sphing-4-enine-1-phosphocholine (N12:0-sphingomyelin) as the internal standard and standard curves of variable amounts of different sphingomyelin molecular species (N16:0-N24:0) versus fixed amount of the internal standard.

### Results

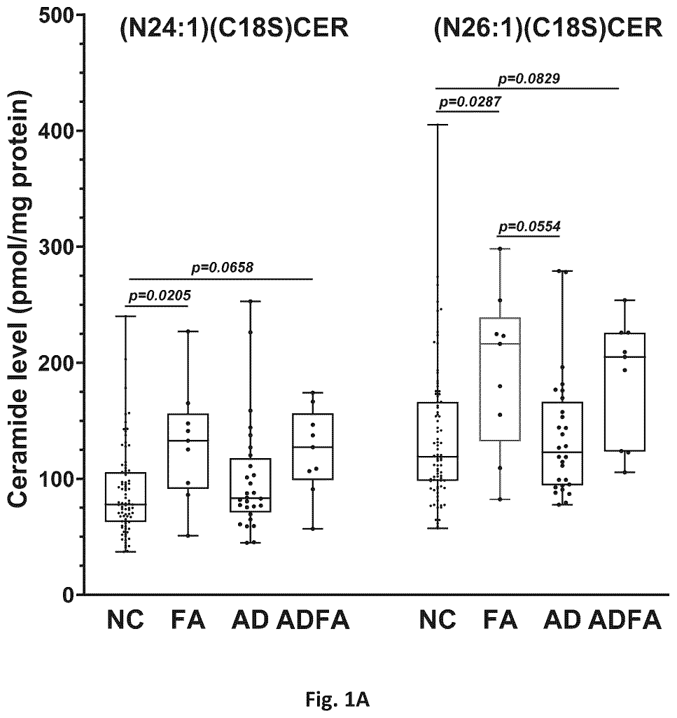

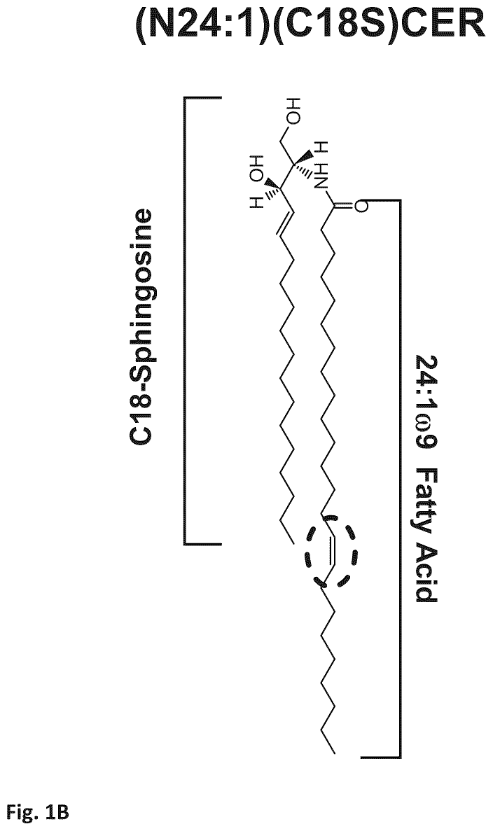

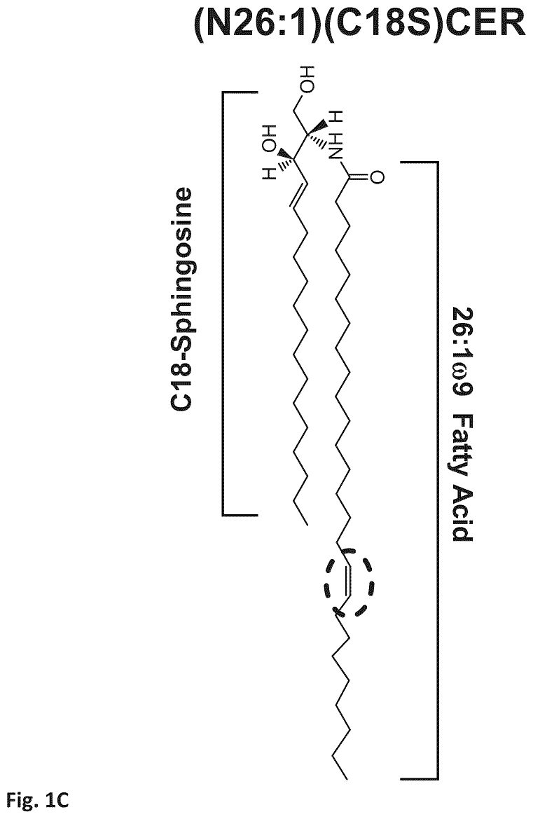

Overall, 8/119 (6.7%) and 10/119 (8.4%) infants developed FA (egg white, peanut, milk) and FA with AD within two years of life, respectively. Lipidomics identified FA-specific increase in the content of NS-ceramides with C18-sphingosine and long-chain monounsaturated fatty acids N24:1 (N24:1(C18S) CER, mean, pmol/mg protein, FA vs healthy: 140, 89, p=0.0047) and N26:1 (N26:1(C18S) CER, mean, pmol/mg protein, FA vs healthy: 204, 138, p=0.163). Infants with future FA and AD (26/119 infants (21.8%)) also had increased levels of N24:1(C18S) CER and N26:1(C18S) CER versus healthy infants (p=0.0032 and 0.0264, correspondingly). Infants with future AD without FA had unchanged levels of N24:1(C18S) CER and N26:1(C18S) CER versus healthy infants (p=0.941 and 0.947, correspondingly). See FIG. 1A.

### Example 2

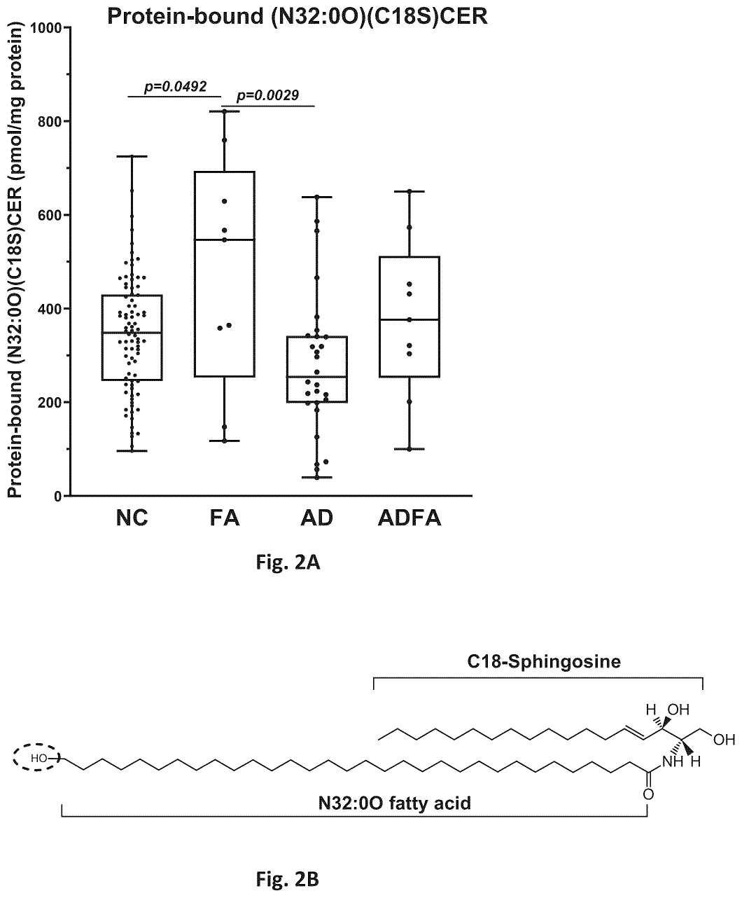

Using the methods as discussed in Example 1, future onset of FA was also found to be accompanied by the increase in protein-bound ceramides ((N32:0O) (C18S)-CER) while AD was characterized by a decrease in protein-bound ceramides (FIG. 2A).

### Example 3

This example is an expansion of the experiments and results disclosed in Example 1 and Example 2 above.

Clinical information: All infants were divided into the control and risk groups for the development of FA and atopic dermatitis (AD). At enrollment, parents completed a questionnaire to provide demographic information and underwent a skin prick test (SPT) for common inhalant allergens. The risk group was identified based on family history of allergic diseases and SPT response, when they met 1 of 2 criteria: (1) at least one parent with both a positive skin test response and a history of asthma or allergic rhinitis, or (2) at least one parent or sibling with physician-diagnosed AD. The control group comprised of infants whose parents had neither an allergy history nor a positive SPT response. In this birth cohort study, we enrolled 87 infants in the risk group and 42 infants in the control group.

All infants underwent regular follow-up assessments at 2, 6, 12, and 24 months of age to monitor for AD and FA. The diagnosis of AD was based on Hanifin and Rajka's criteria (Silverberg, NB. Clin Dermatol 2017; 35:354-9). FA was confirmed by a positive oral food challenge test or a convincing history of adverse reactions within 2 hours of food ingestion plus positive serum specific IgE (≥0.35 kU/L) determined by the Immuno-CAP system (ThermoFisher Scientific, Waltham, MA, USA). When the parents reported any suspicious allergic symptoms, the infants were brought to the outpatient clinic and examined by pediatric allergists. Immediate reactions included urticaria, eyelid edema, lipedema, cough, wheezing, vomiting, diarrhea, abdominal pain, hypotension, and altered mentality. Table 1 provides the clinical characteristics of the subjects:

TABLE 1
Clinical characteristics of subjects

| | | Control group | |
|---|---|---|---|
| | Food allergy | NC (n = 83) and | |
| | (FA and ADFA) | AD (n = 28) | |
| | (n = 18) | (n = 111) | P value |
| Sex (male) | 13 *72.2%) | 61 (55.0%) | 0.169 |
| Family history of allergic diseases | 13 (72.2%) | 74 (66.7%) | 0.641 |
| Birth type (Cesarean section) | 6 (33.3%) | 45 (40.5%) | 0.562 |
| Preterm birth | 1 (5.6%) | 6 (5.4%) | 1.000 |
| Presence of atopic dermatitis during 2 years of age | 9 (50.0%) | 28 (25.2%) | 0.031 |
| Use of systemic antibiotics at age <6 mo | 5 (27.8%) | 17 (15.3%) | 0.192 |
| Breastfeeding during the first 6 mo | 8 (44.4%) | 57 (51.4%) | 0.587 |
| Introduction of solid foods before 6 mo11 (61/1%) | 4 (22.2%) | 30 (27.0%) | 0.780 |
| Monthly income (>4000 US $) | 11 (61.1%) | 79 (71.2%) | 0.389 |
| Maternal education (College) | 16 (88.9%) | 96 (86.5%) | 1.000 |
| Vitamin D supplementation during pregnancy | 7 (38.9%) | 59 (53.2%) | 0.261 |

### STS Analyses:

STS, layers 5 and 6, were subjected to the processing protocol and liquid chromatography tandem mass spectrometric analysis (Berdyshev E, et al. Methodological Considerations for Lipid and Polar Component Analyses in Human Skin Stratum Corneum. Cell Biochem Biophys 2021; 79:659-68; Berdyshev E, et al. Dupilumab significantly improves skin barrier function in patients with moderate-to-severe atopic dermatitis. Allergy 2022; 77:3388-97) that identified the following groups of sphingolipids: sphingoid bases, sphingomyelins (SM, with C18-sphingosine), NS-ceramides (C14-C32 non-hydroxy fatty acids and C18-, C20-, C22-sphingosine), NDS-ceramides (non-hydroxy fatty acids and C18-, C20-, C22-dihydrosphingosine), NP-ceramides (C14-C32 non-hydroxy fatty acids and C18-, C20-, C22-phytosphingosine), AS-ceramides (C14-C32 alpha-hydroxy fatty acids and C18-, C20-, C22-sphingosine), EOS-ceramides (esterified omega-hydroxy fatty acid containing ceramides with C18-, C20-, C22-sphingosine), and protein-bound OS-ceramides (with omega-hydroxy fatty acids and C18-, C20-, C22-sphingosine). Relative percentages within NS-ceramide groups were calculated accounting for the absolute amounts of NS-ceramides with 14:0, 16:0, 18:0, 20:0, 22:0, 24:1, 24:0, 26:1, 26:0, 28:0, 30:0, and 32:0 fatty acids within each individual subgroup of NS-ceramides (with C18-, C20-, or C22-sphingosine). Similarly, relative percentages of SM molecular species were calculated, accounting for the absolute amounts of SM with C18-sphingosine and 14:0, 16:0, 18:0, 20:0, 22:0, 24:1, 24:0, 26:1, 26:0, and 28:0 fatty acids. gamma-induced protein 10 (IP-10). Further details of skin prick test (SPT), skin tape stripping, protein extraction, lipid mass spectrometry analysis, and Cytokine MSD assay can be found in Berdyshev E, et al. Stratum corneum lipid and cytokine biomarkers at age 2 months predict the future onset of atopic dermatitis. J Allergy Clin Immunol 2023; 151:1307-16.

Statistical analysis: Data were analyzed using SAS software, version 9.4 (SAS Institute Inc., Cary, NC, USA.), R software, version 4.2.2 (R Project for Statistical Computing), and GraphPad Prism (version 10.0.2, GraphPad Software, San Diego, CA, USA). The Chi-squared test or Fisher's exact test was applied to determine the differences in the proportions. Epidermal lipid and cytokine levels were compared using ANOVA with correction for multiple comparisons (Tukey).

Logistic regression analysis was conducted to determine the effects of cytokines and lipids on FA development. For each of the cytokines and lipids at the age of 2 months, the receiver operating characteristic curve was analyzed to examine its diagnostic values and determine an optimal cutoff point when predicting the development of FA. The optimal cutoff point was identified by maximizing the balanced accuracy via the Youden index method. Binary variables, indicating high-risk versus low-risk levels, were defined based on these cutoff points for each cytokine and lipid. The individual and combined effects of cytokines and lipids were assessed using odds ratios estimated from logistic regression models. Firth's penalized method was applied if needed to reduce possible sparsity issues. AD was not adjusted for in the analysis of FA development, as our focus was on investigating the effects of lipids and cytokines on both AD and FA separately. The cutoff values determined by receiver operating characteristic (ROC) assessment based on the STS data at 2 months for FA prediction at 24 months and the proportion of study subjects in the low- and high-risk groups based on the ROC cut-offs for the cytokine and lipid predictors of FA development are shown in Tables 2 and 3, respectively. A P value <0.05 was considered to be significant.

TABLE 2
Cutoff values determined by ROC assessment based on the skin tape
strip data at 2 months for food allergy prediction at 24 months

| Variables | Cutoff value | |
|---|---|---|
| IL-13 | >23 | pg/mg protein |
| TNF-α | >3 | pg/mg protein |
| IL-33 | >460 | pg/mg protein |
| 24:1-SM | ≤0.1 | pmol/mg/protein |
| 26:1-SM | ≤0.7 | pmol/mg/protein |
| (N32:0O)(C18S)CER | >540 | pmol/mg/protein |

| | |
|---|---|
| % N24:1(C18S)CER within N(C18S)CER | >9.7% |
| % N26:1(C18S)CER within N(C18S)CER | >14.4% |
| % N24:1(C20S)CER within N(C20S)CER | >6.6% |
| % N24:1(C20S)CER within N(C20S)CER | >10.1% |

TABLE 3
The proportion of study subjects in the low- and high-risk groups based on
the ROC cut-offs for the cytokine and lipid predictors of FA development.

| | No. of no FA subjects | | No. of FA patients | |
|---|---|---|---|---|
| | (NC + AD, proportion*) | | (FA + ADFA, proportion*) | |
| Variables | Low-risk | High-risk | Low-risk | High-risk |
| IL-13 | 83 (74.8%) | 28 (25.2%) | 5 (27.8%) | 13 (72.2%) |
| TNF-α^{‡} | 74 (76.3%) | 23 (23.7%) | 4 (22.2%) | 14 (77.8%) |
| IL-33 | 100 (90.1%) | 11 (9.9%) | 11 (61.1%) | 7 (38.9%) |
| 24:1-SM | 102 (91.9%) | 9 (8.1%) | 10 (55.6%) | 8 (44.4%) |
| 26:1-SM | 95 (85.6%) | 16 (14.4%) | 10 (55.6%) | 8 (44.4%) |
| (N32:0O)(C18S)CER | 104 (93.7%) | 7 (6.3%) | 11 (61.1%) | 7 (38.9%) |
| % N24:1(C18S)CER within N(C18S)CER | 88 (79.3%) | 23 (20.7%) | 5 (27.8%) | 13 (72.2%) |
| % N26:1(C18S)CER within N(C18S)CER | 91 (82.0%) | 20 (18.0%) | 7 (38.9%) | 11 (61.1%) |
| % N24:1(C20S)CER within N(C20S)CER | 65 (58.6%) | 46 (41.4%) | 2 (11.1%) | 16 (88.9%) |
| % N24:1(C20S)CER within N(C20S)CER | 72 (64.9%) | 39 (35.1%) | 6 (33.3%) | 12 (66.7%) |

*Percentages indicate the proportion of subjects in the low- and high-risk groups out of total subjects for NC + AD group or FA + ADFA group for each variable.
^{‡}TNF-α values are based on 115 subjects due to 14 missing data.

### Results:

Out of 129 newborns, 9 children developed FA without AD, another 9 children developed ADFA, and 28 infants developed AD without FA during the first two years of life, with 83 infants staying healthy (Table 1). The most common food allergen was egg white (16/18); one child each had an allergy to walnut and cow's milk. Two children had an allergy to peanuts in addition to egg white, and one infant had an allergy to egg white, peanuts, and walnuts. At the age of two months, when the SC was collected by skin tape stripping, none of the infants had a history of clinical FA or AD.

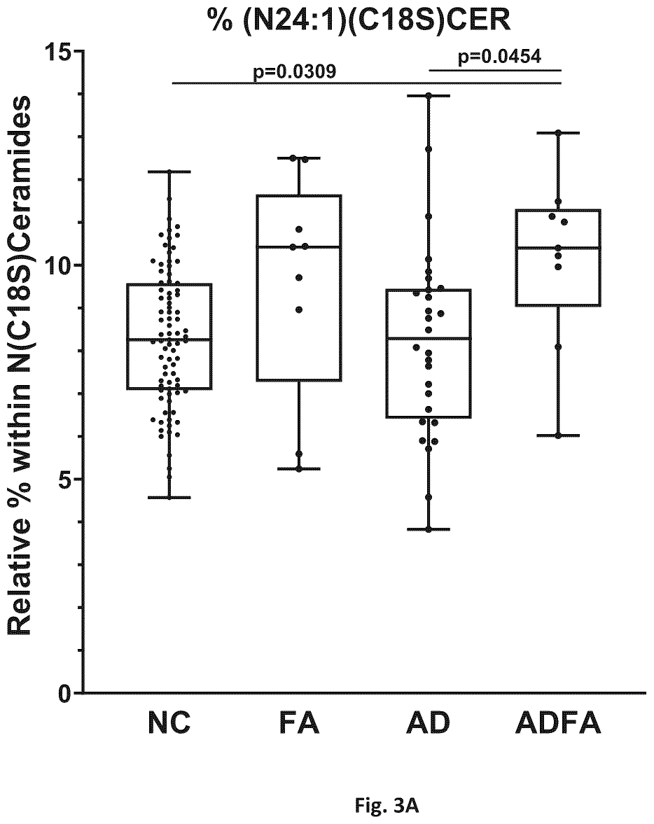

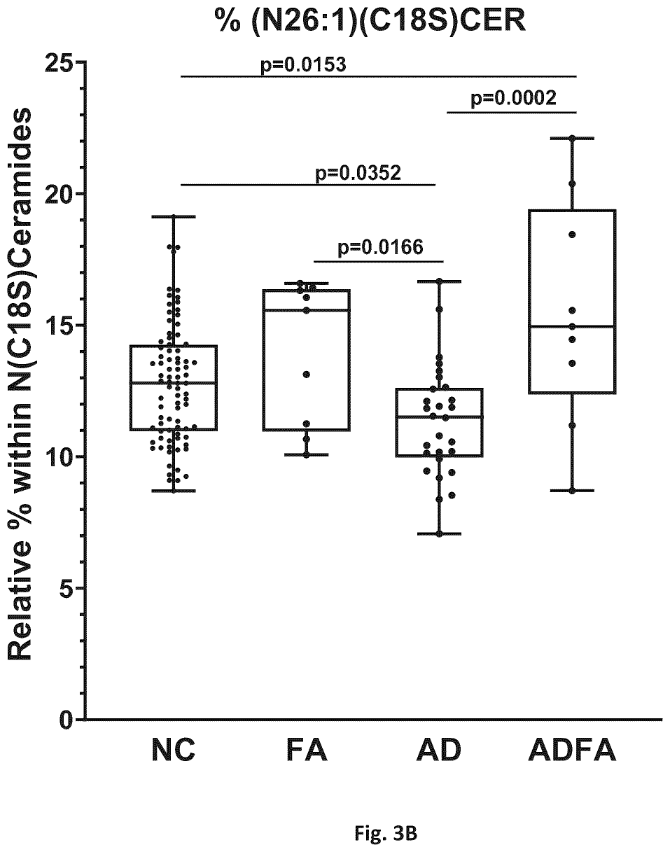

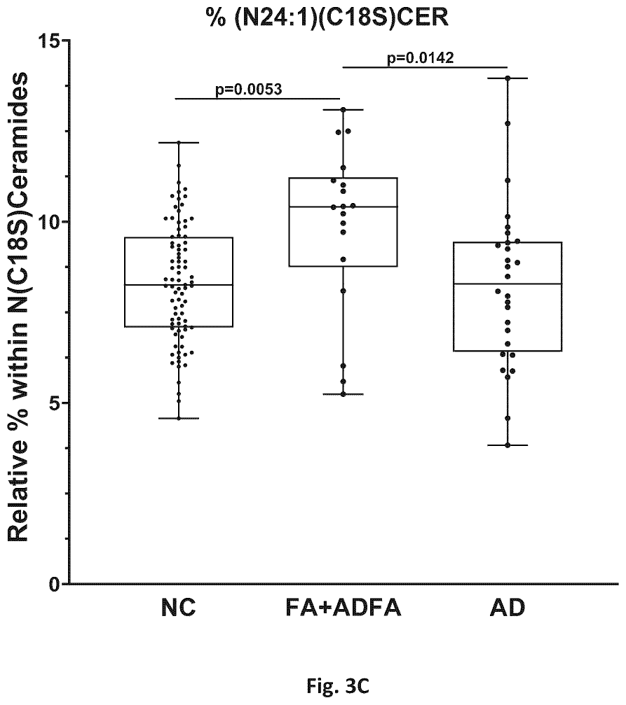

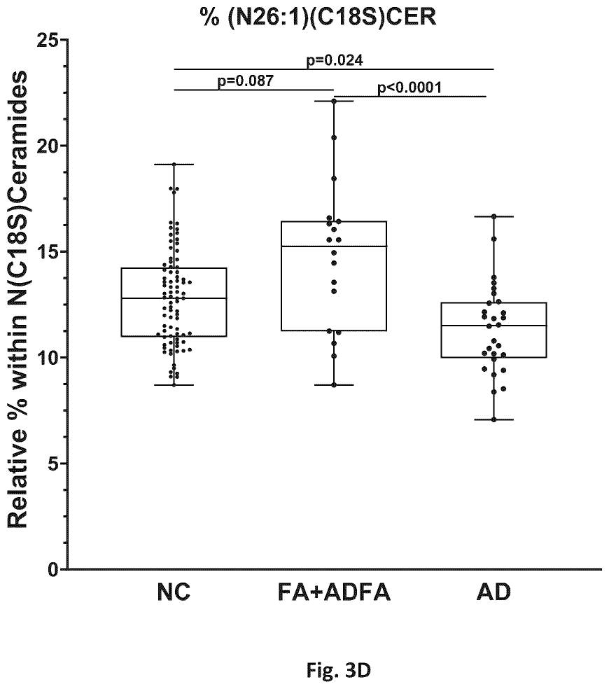

The detailed analysis of lipids in the SC of infants who later in life developed FA revealed unique lipid abnormalities that separated them from healthy infants as well as from infants who developed only AD later in life. In particular, the SC of future FA infants had increased levels (FIG. 1A) and proportions (FIGS. 3A-3B) of N(C18S)-ceramides with unsaturated fatty acids (N24:1 and N26:1, here and thereafter, amide-linked fatty acids, carbon chain length:number of double bonds) and C18-sphingosine. Infants who developed both FA and AD later in their lives also had increased levels and proportions of these unsaturated NS-ceramides, while children with future AD only were not different from healthy children for this lipid parameter. This signifies that such an increase in NS-ceramide unsaturation is unique to FA and not to AD. Combining FA and ADFA infants into one group further strengthened the difference between FA, AD, and healthy groups (FIGS. 3C-3D).

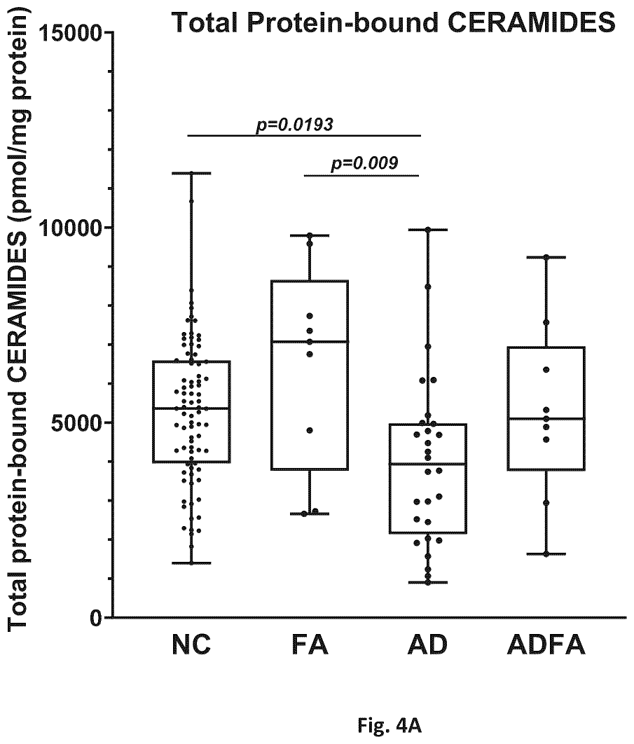

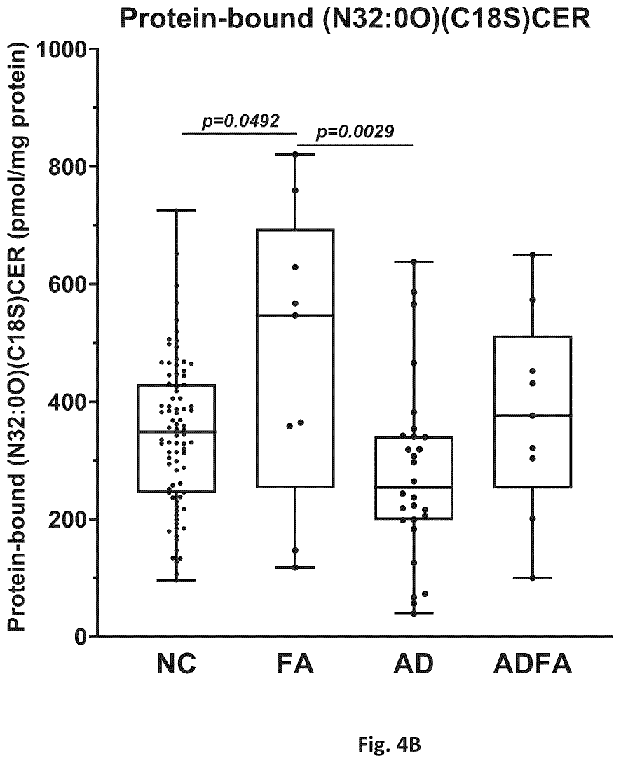

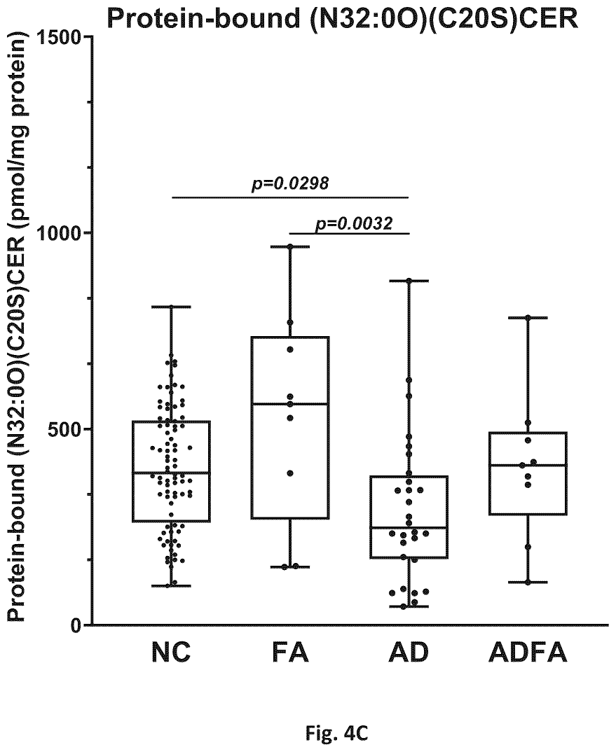

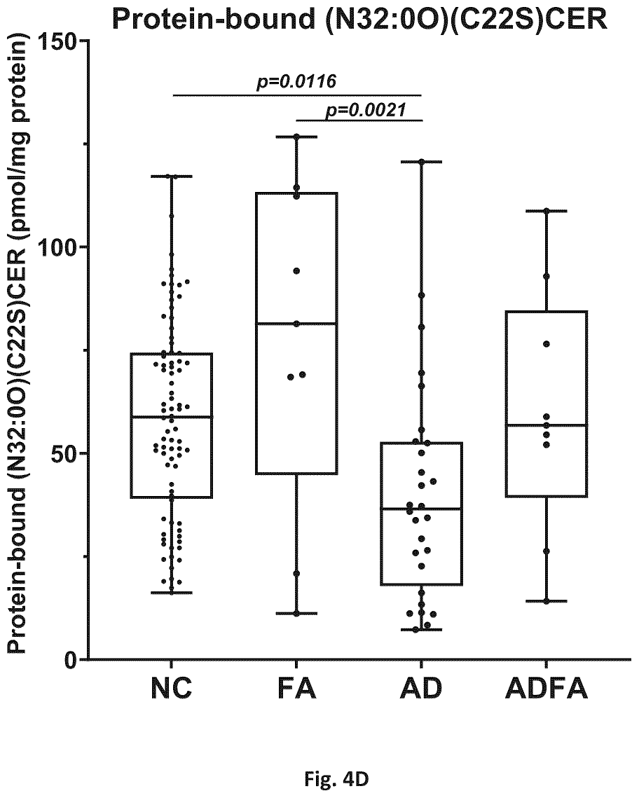

Protein-bound OS-ceramides are another group of SC lipids that may help to identify future FA children. In contrast to infants with future AD who had decreased levels of OS-ceramides, the SC of future FA infants had a tendency to have increased levels of OS-ceramides as compared to healthy skin (FIG. 4A). This increase, while found so far to be statistically significant only for one of the OS-CER molecular species (with the longest measured fatty acid (N32:0O) (C18S) CER)) (FIG. 4B), contrasted to an overall decline in OS-ceramides in future AD infants as compared to SC of healthy infants. At two months of age, in the skin of future FA children, the levels of N32:0O—S-ceramides with all three sphingoid bases (C18-C22) and total levels of protein-bound OS-ceramides (the sum of twelve measured molecular species) were significantly increased as compared to the skin of children who later developed AD (FIGS. 4B-4D). This finding has to be confirmed in other studies with larger numbers of future FA infants, but already indicates that there is no impairment in biosynthesis of OS-ceramides in the SC of future FA infants. Such an increase in OS-ceramides, especially in the most hydrophobic ultra-long-chain OS-ceramide species, might be a compensatory mechanism to counteract the increase in unsaturated NS-ceramide species that are known to impair barrier properties of lipid ultrastructures by reducing the packing density and enhancing the permeation and water loss (Mojumdar E H, et al. Monounsaturated fatty acids reduce the barrier of stratum corneum lipid membranes by enhancing the formation of a hexagonal lateral packing. Langmuir 2014; 30:6534-43). Interestingly, in future FA infants with concomitant AD, FA and ADFA metabolically counteract each other, leading to a net no change in OS-ceramides (FIGS. 4A-4D).

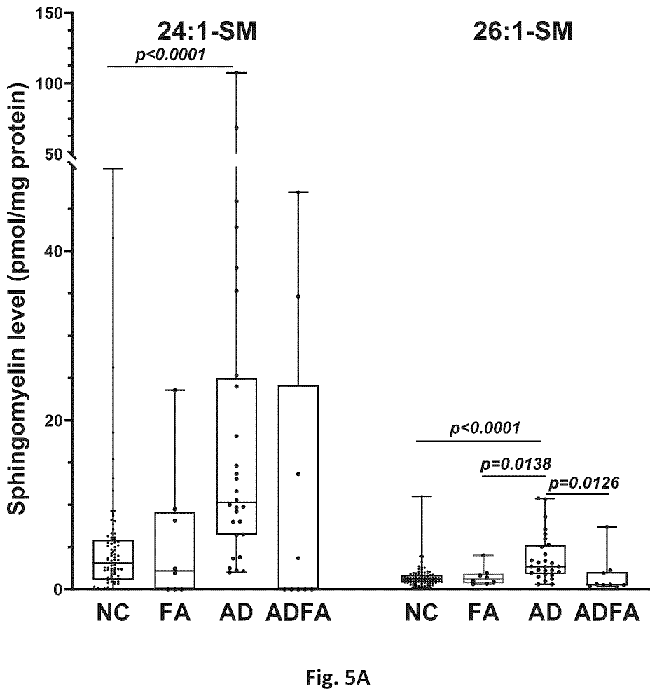

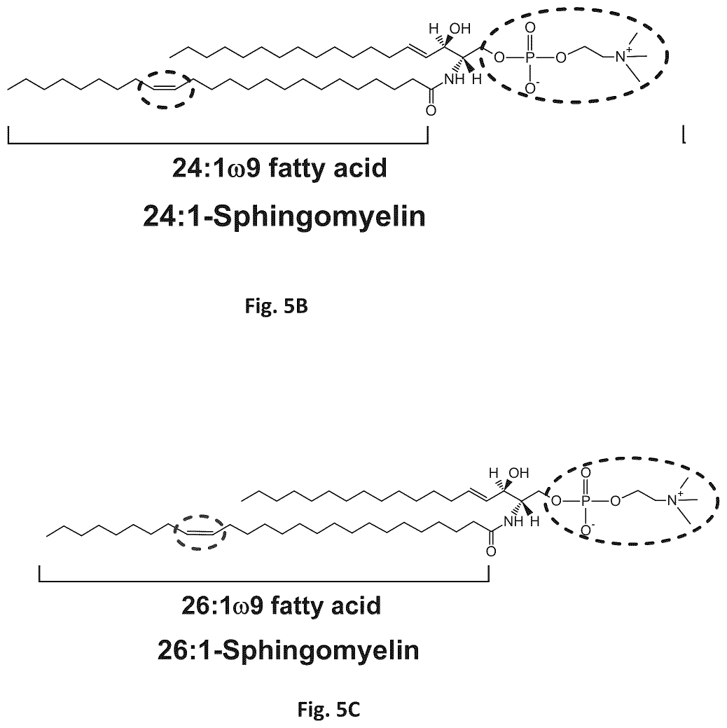

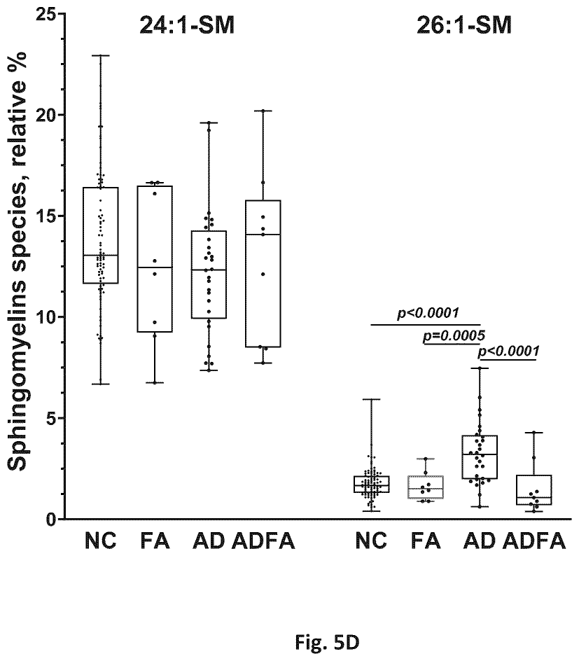

Previously, the inventors observed that the SC of infants with future AD onset demonstrates an increase of unsaturated sphingomyelin (SM) species with 24:1 and 26:1 fatty acids (Berdyshev E, et al. Stratum corneum lipid and cytokine biomarkers at age 2 months predict the future onset of atopic dermatitis. J Allergy Clin Immunol 2023; 151:1307-16). Remarkably, infants with future onset of FA or ADFA have no increase in such SM molecular species (FIG. 5A and FIG. 5D) in SC. Such a dichotomy in the distribution of 24:1 and 26:1 fatty acids between N(C18S)-ceramides and sphingomyelins between infants with future AD only and those with future onset of FA or ADFA suggests a unique, AD-specific mechanism of regulation of SM biosynthesis in the skin that is counteracted and abolished by the development of FA. Currently, there is no clear understanding of how ceramide redistribution between biosynthesis of SM and glycosphingolipids is regulated. Ceramides are the immediate precursors for both of these complex sphingolipids. However, the biosynthesis of SM and glycosphingolipids occurs in trans-Golgi, while ceramides are synthesized in the endoplasmic reticulum and need to be transported to cis-Golgi by a CERT carrier protein (Kumagai K, et al. Structure, functions and regulation of CERT, a lipid-transfer protein for the delivery of ceramide at the ER-Golgi membrane contact sites. FEBS Lett 2019; 593:2366-77). Therefore, multiple potential targets involved in ceramide transport and complex sphingolipid biosynthesis should be further explored as differentially controlled in FA versus AD skin.

All of the documents cited herein are incorporated herein by reference.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed is:

1. A method of treating a human infant identified as being at risk of developing a food allergy (FA), the method comprising:
a. determining level of one or more unsaturated N-ceramides with C18-sphingosine (N(C18S)CERs), one or more protein-bound OS-ceramides, interleukin-33 (IL-33) or combination thereof in a non-lesional skin sample from the infant;
b. comparing the level of the one or more N(C18S)CER, the protein-bound OS-ceramide, IL-33 or a combination thereof in the skin sample from the infant to a level of the same one or more N(C18S)CER, protein-bound OS-ceramide, IL-33 or combination thereof from a control;
wherein an elevated level of the one or more N(C18S)CER, protein-bound OS-ceramide, IL-33, or combination thereof in the skin sample from the infant as compared to the control level identifies the infant as being at risk of developing a FA; and
treating the infant identified as being at risk of developing a FA with one or more therapeutic strategies selected from the group consisting of early introduction of one or more allergenic foods, implementation of a diversified diet comprising one or more allergenic foods, prevention/reduction of household exposure to one or more allergenic foods, administration of a skin barrier enforcing emollient, and elimination of S. aureus colonization and infection.

2. A method to prevent the onset of FA symptoms and/or reduce the severity and/or delay the onset of FA symptoms in a human infant at risk of developing a FA, the method comprising:
a. determining level of one or more unsaturated N-ceramides with C18-sphingosine (N(C18S)CERs), one or more protein-bound OS-ceramides, IL-33 or combination thereof in a non-lesional skin sample from the infant; and
b. comparing the level of the one or more N(C18S)CER, protein-bound OS-ceramide, IL-33, or combination thereof in the skin sample from the infant to a level of the same one or more N(C18S)CER, protein-bound OS-ceramide, IL-33 or combination thereof from a control;
wherein an elevated level of the N(C18S)CER, protein-bound OS-ceramide, IL-33 or combination thereof in the skin sample from the infant as compared to the control level identifies the infant as being at risk of developing a FA;
wherein the infant identified as being at risk of developing a FA undergoes one or more therapeutic strategies and/or preemptive interventions selected from the group consisting of early introduction of one or more allergenic foods, implementation of a diversified diet comprising one or more allergenic foods, prevention/reduction of household exposure to one or more allergenic foods, administration of a skin barrier enforcing emollient, elimination of S. aureus colonization and infection, allergen-specific immunoglobulin E (IgE) testing to one or more food allergens, skin prick testing (SPT) to one or more food allergens, and an oral food challenge to one or more food allergens.

3. A method of preemptive intervention in a human infant at risk of developing a FA, the method comprising:
a. determining level of one or more unsaturated N-ceramides with C18-sphingosine (N(C18S)CERs), one or more protein-bound OS-ceramides, IL-33 or combination thereof in a non-lesional skin sample from the infant; and
b. comparing the level of the one or more N(C18S)CER, protein-bound OS-ceramide, IL-33 or combination thereof in the skin sample from the infant to a level of the same N(C18S)CER, protein-bound OS-ceramide, IL-33 or combination thereof from a control;
wherein an elevated level of the N(C18S)CER, protein-bound OS-ceramide, IL-33 or combination thereof in the skin sample from the infant as compared to the control level identifies the infant as being at risk of developing a FA;
wherein the infant identified as being at risk of developing a FA undergoes one or more preemptive interventions selected from the group consisting of early introduction of one or more allergenic foods, implementation of a diversified diet comprising one or more allergenic foods, prevention/reduction of household exposure to one or more allergenic foods, administration of a skin barrier enforcing emollient, elimination of S. aureus colonization and infection, allergen-specific immunoglobulin E (IgE) testing to one or more food allergens, skin prick testing (SPT) to one or more food allergens, and an oral food challenge to one or more food allergens.

4. The method of any one of claims 1-3, wherein the infant has not been diagnosed as having an atopic disease.

5. The method of any one of claims 1-3, wherein the infant has a family history of an atopic disease.

6. The method of claims 1-3, wherein the one or more N(C18S)CER comprise a long-chain monounsaturated fatty acid.

7. The method of claim 6, wherein the one or more N(C18S)CER is selected from the group consisting of N24:1 (C18S) CER, N26:1 (C18S) CER, and combinations thereof.

8. The method of any one of claims 1-3, wherein the protein-bound OS-ceramide is an N32:0O—S-ceramide.

9. The method of claim 8, wherein the N32:0O—S-ceramide is (N32:0O) (C18S) CER.

10. The method of claim 1, wherein the infant identified as being at risk of developing a FA undergoes testing to confirm and/or determine if the infant has a FA.

11. The method of any one of claims 1-3, wherein the infant identified as being at risk of developing a FA further develops atopic dermatitis (AD).

12. The method of any one of claims 1-3, wherein the skin sample is obtained by a skin tape stripping method.

13. The method of claim 12, wherein the skin tape stripping method comprises:
a. applying an adhesive tape to a target area of the skin of the infant in a manner sufficient to isolate an epidermal sample adhering to the adhesive tape, wherein the epidermal sample comprises the one or more N(C18S)CERs, one or more protein-bound OS-ceramides, IL-33 or combination thereof from the stratum corneum of the infant, wherein the tape comprises a polymeric adhesive; and
b. extracting the epidermal sample comprising the one or more N(C18S)CERs, one or more protein-bound OS-ceramides, IL-33 or combination thereof adhering to the adhesive tape with a cell scraper comprising thermoplastic elastomer material in a solvent of about 5% to about 30% alcohol in water.

14. The method of claim 13, further comprising determining the expression level of the one or more N(C18S)CER, one or more protein-bound OS-ceramide, IL-33 or combination thereof in the epidermal sample.

15. The method of any one of claims 1-3 further comprising determining thymic stromal lymphopoietin (TSLP) level in the skin sample from the infant and comparing the level to TSLP level in the control sample, wherein the level of TSLP in the infant as compared to the TSLP level of the control is not elevated.

16. The method of any one of claims 1-3 further comprising determining the level of TNF-alpha, interleukin-13 (IL-13), interleukin-6 (IL-6) and/or MDC in the infant and comparing the level to levels of TNF-alpha, interleukin-13 (IL-13), interleukin-6 (IL-6) and/or MDC from the control.
